(19) United States
(12) Patent Application Publication
(10) Pub. No.: US 2014/0261462 A1
(43) Pub. Date: Sep. 18, 2014

(54) Endotracheal Tube Holding Device with Bite Block

(71) Applicant: Hollister Incorporated, (US)

(72) Inventors: Peter L. Visconti, Gurnee, IL (US); Brian T. Leadingham, Pleasant Prairie, WI (US); Patrick C. Tetzlaff, Racine, WI (US); Paola M. Wisner, Des Plaines, IL (US); Richard J. Hantke, Chicago, IL (US); Meagan R. Shanahan Pheil, Chicago, IL (US); David A. Knauz, Riverwoods, IL (US); Cheryl D. Berger, Round Lake, IL (US); George J. Cisko, Spring Grove, IL (US); Thai H. Dang, Gurnee, IL (US); Thomas H. Gilman, Spring Grove, IL (US); Noah K. Meade, Grayslake, IL (US); Tze Wan Pansy Chung, Fox River Grove, IL (US); Christina Augustyn, Chicago, IL (US); Daniel A. March, Lake Villa, IL (US); David McDonough, Pleasant Prairie, WI (US)

(73) Assignee: Hollister Incorporated, Libertyville, IL (US)

(21) Appl. No.: 13/840,375

(22) Filed: Mar. 15, 2013

(51) Int. Cl. A61M 16/04; A61C 5/14

(52) U.S. Cl. CPC A61M 16/0497; A61C 5/14
USPC 128/861

(57) ABSTRACT

A device for holding an endotracheal tube has a slide track configured to fit adjacent a lip on a patient's face. The track has a face contacting side, an exposed side, and a pair of opposite ends. A tube holder is coupled to and slidable along the track. A positioning mechanism is releasably lockable to allow selective lateral repositioning of the tube holder and an endotracheal tube held thereby along the track and to retain the tube holder at a selected position along the track without removing the device from the patient or an endotracheal tube from the device. A bite block is carried by the tube holder and slidable therewith along the track. The bite block has a tubular wall with a central opening along a length of the bite block and a pair of opposite open ends. The bite block is positioned spaced vertically from the track and has a portion extending rearward beyond the face contacting side of the track.

### BACKGROUND

1. Field of the Disclosure

The present invention is generally directed to medical tube devices, and more particularly to a device for holding an endotracheal tube on a patient and having a bite block integrated with the device.

2. Description of Related Art

Endotracheal (ET) tubes are commonly inserted through the mouth and into the trachea of patients under critical care. The ET tube is used to maintain an open airway for the patient to breathe and to allow mechanical assistance of breathing. ET tubes are often placed prior to surgery or are used on trauma or critically ill patients that may require intubation for extended periods of time. Many instances in which a patient is intubated require that the tube remain in place for approximately 48 to 72 hours and, in some circumstances, the period of use may be extended for 7 to 14 days or more

There are many known methods and devices for securing an ET tube on a patient. One such device is manufactured and sold by Hollister, Inc., the assignee of the present patent under the name of Anchor Fast. The Anchor Fast product has a track that is secured to a patient via a head strap. The track is connected to cheek plates with cheek pads that are skin friendly and that have adhesive patches to help retain the track in the proper position on the patient's face during use. A tube holder is side-to-side adjustable on the track and is used to secure an ET tube to the device.

An earlier version of the Anchor Fast ET tube holder is disclosed in U.S. Pat. No. 5,490,504 to Hollister, Inc. The '504 patent discloses a device that has the track and tube holder, including side-to-side adjustability of the tube holder and ET tube secured thereto. However, the '504 patent does not include the cheek pads and cheek plates.

One problem with these and other known ET tube holding devices is that the patient may sometimes bite down hard or clamp onto the ET tube with their teeth or gums (denture wearers). This can restrict or close off the airway within the tube. Some manufacturers have developed stand-alone bite blocks that can be attached to the ET tube and placed between the patient's teeth during use. These types of bite block products must be separately manufactured, distributed, stored, and installed. These types of bite blocks are positioned around the ET tube and are intended to protect the ET tube and inhibit crushing of the ET tube by the patient. Others have attempted to develop air assist breathing devices and even ET tube holders that incorporate a bite block function into the product. These known products are generally large, cumbersome, and complicated devices that can be difficult to manipulate and install. Medical personnel have not heretofore taken to these types of integrated products.

Another problem with these types of ET tube holding devices is that the tube often is accompanied by one or more accessory lines that also pass into the patient. For example, many ET tubes have an inflation cuff on the end of the tube that is inserted into the patient. The cuff is inflated after insertion of the tube to help retain the ET tube in the patient's trachea. Typically, a pilot line is coupled to the cuff and extends along the outside of the ET tube. The pilot line routing must be taken into account so that the line is not crushed or pinched off by any part of the holding device or the ET tube. This is particularly true where a bite block function is provided.

### SUMMARY

In one example according to the teachings of the present invention, a device for holding an endotracheal tube to a patient has a track configured to fit adjacent a lip on a patient's face. The track has a face contacting side, an exposed side opposite the face contacting side, and a pair of opposite ends. A tube holder is coupled to and slidable along the track between the opposite ends. A positioning mechanism is releasably lockable to allow selective lateral repositioning of the tube holder and an endotracheal tube held thereby along the track and to retain the tube holder at a selected position along the track. A bite block is carried by the tube holder and is slidable therewith along the track. The bite block has a tubular wall with a generally cylindrical shape, a central opening along a length of the bite block, and a pair of opposite open ends. The bite block is positioned and spaced vertically from the track and has a working end portion that extends in the direction of and further beyond the face contacting side of the track.

In one example, the bite block can have an insertion slot through the wall and along the entire length of the bite block. The wall can be being sufficiently flexible to allow an endotracheal tube to be inserted laterally into the central opening through the insertion slot.

In one example, the bite block can have an insertion slot through the wall and along the entire length of the bite block. The insertion slot can be defined between spaced apart free edges of the wall that confront one another along the length of the bite block.

In one example, one of the free edges can have a first contoured shape and the other of the free edges can have a corresponding second contoured shape configured to close against and engage the first contoured shape if a sufficient clamping force is exerted on an outer surface of the bite block, such as if a critical care patient were to bite down on the bite block during use.

In one example, one of the free edges can have a convex rounded edge and the other of the free edges can have a concave rounded edge sized to receive the convex rounded edge.

In one example, one of the free edges can have an angled or beveled edge and the other of the free edges can have a corresponding angled or beveled edge arranged to engage and mate with one another.

In one example, one of the free edges can have a convex V-shaped protrusion and the other of the free edges can have a corresponding concave V-shaped groove configured to receive the convex V-shaped protrusion.

In one example, each of the free edges can have a flange extending inward into the central opening of the bite block and can be configured to close against and engage one another if a sufficient clamping force is exerted on an outer surface of the bite block.

In one example, each of the free edges can have a flange extending inward into the central opening of the bite block and can be configured to close against and engage one another. An accessory line channel can be formed or defined along an inner surface of the wall adjacent one or both of the flanges within the central opening of the bite block.

In one example, the bite block can have an insertion slot through the wall and along the entire length of the bite block. A living hinge or relief can be provided in the wall generally opposite the insertion slot and extending a length of the bite block. The living hinge can render the wall sufficiently flexible to allow an endotracheal tube to be inserted laterally into the central opening through the insertion slot.

In one example, a blind slot can be formed in the working end portion of the bite block and can extend only part way along the length of the wall of the working end portion.

In one example, the bite block can have an insertion slot through the wall and along the entire length of the bite block. A blind slot can be formed in the working end portion of the bite block and can extend only part way along the length of the wall of the working end portion and can be positioned generally opposite the insertion slot

In one example, the device can have an accessory line channel extending the length of the bite block and formed between an inner surface of the wall and an outer surface of an endotracheal tube positioned within the central opening of the bite block.

In one example, an accessory line channel can be formed between two spaced apart ribs extending the length of the bite block on an inner surface of the wall.

In one example, an accessory line channel can be formed as a V-shaped groove extending along and recessed into an inner surface of the wall.

In one example, the track can have a lip pad on the face contacting side.

In one example, the track can be configured to rest above an upper lip of a patient and the bite block can be spaced below the track.

In one example, the device can include a cheek plate connected to each of the opposite ends of the track, a cheek pad coupled to each of the cheek plates, an adhesive layer on a face contacting side of each pad, and an adjustable head strap coupled to the device for securing the device to a patient's head and retaining the track, cheek plates, and cheek pads on the patient's face.

In one example, the tube holder can have a tube strap configured to wrap around and secure an endotracheal tube to the tube holder.

In one example, the tube holder can have a tube strap configured to wrap around and secure an endotracheal tube to the tube holder. A surface of the tube strap can include an adhesive that contacts an outer surface of an endotracheal tube when the tube strap is wrapped around the endotracheal tube.

In one example, the device can include retention spikes protruding from a surface of the tube holder and positioned to contact and impinge on an outer surface of an endotracheal tube when attached to the tube holder.

In one example according to the teachings of the present invention, an endotracheal tube securing device has a track configured to fit above an upper lip on a patient's face. The track has a face contacting side, an exposed side opposite the face contacting side, and a pair of opposite ends. A tube holder is coupled to and slidable along the track between the opposite ends. A positioning mechanism is releasably lockable to allow selective lateral repositioning of the tube holder and an endotracheal tube held thereby along the track and to retain the tube holder at a selected position along the track. An adjustable head strap or multiple straps are coupled to the device for securing the device to a patient's head and retaining the track on the patient's face. A cheek plate is connected to each of the opposite ends of the track. Each cheek plate carries a cheek pad with an adhesive layer on a face contacting side of the cheek pad. A bite block is integrally molded as part of the tube holder and is slidable therewith along the track. The bite block has a tubular wall with a generally cylindrical shape, a central opening along a length of the bite block, and a pair of opposite open ends. An insertion slot is formed through the wall and along the entire length of the bite block. The bite block is positioned spaced vertically below the track and has a working end portion extending further rearward than a plane of the face contacting side of the track.

### BRIEF DESCRIPTION OF THE DRAWINGS

Objects, features, and advantages of the present invention will become apparent upon reading the following description in conjunction with the drawing figures, in which:

FIG. 1 shows a front and side perspective view of one example of a device for holding or securing an endotracheal tube constructed in accordance with the teachings of the present invention and with a tube holder centered on the device.

FIG. 2 shows a perspective cross-section taken along line 2-2 of the device shown in FIG. 1.

FIG. 3 shows an alternate top, front, and side perspective view of the device shown in FIG. 1 and with the tube holder moved to a position offset from the centered position.

FIG. 4 shows a cross-section taken along line 4-4 of the device shown in FIG. 3.

FIG. 5 shows a rear and side perspective view of only the tube holder portion of the device shown in FIG. 1.

FIG. 6 shows a front view of the tube holder shown in FIG. 5

FIG. 7 shows a rear view of the tube holder shown in FIG. 5.

FIG. 8 shows a right side view of the tube holder shown in FIG. 5.

FIG. 9 shows a top view of the tube holder shown in FIG. 5.

FIG. 10 shows a left side view of the tube holder of FIG. 5.

FIG. 11 shows a bottom view of the tube holder of FIG. 5.

FIG. 12 shows a rear and right perspective view of one alternative example of a tube holder for the device shown in FIG. 1.

FIG. 13 shows a rear view of the tube holder of FIG. 12.

FIG. 14 shows a rear and right perspective view of another alternative example of a tube holder for the device shown in FIG. 1.

FIG. 15 shows a rear view of the tube holder of FIG. 14.

FIG. 16 shows a rear and left side perspective view of another alternative example of a tube holder for the device shown in FIG. 1.

FIG. 17 shows a rear view of the tube holder of FIG. 16.

FIG. 18 shows a rear and left side perspective view of another alternative example of a tube holder for the device shown in FIG. 1.

FIG. 19 shows a rear view of the tube holder of FIG. 18.

FIG. 20 shows a rear and left side perspective view of another alternative example of a tube holder for the device shown in FIG. 1.

FIG. 21 shows a rear view of the tube holder of FIG. 20.

FIG. 22 shows a rear and left side perspective view of another alternative example of a tube holder for the device shown in FIG. 1.

FIG. 23 shows a rear view of the tube holder of FIG. 22.

FIG. 24 shows a rear and left side perspective view of another alternative example of a tube holder for the device shown in FIG. 1.

FIG. 25 shows a rear view of the tube holder of FIG. 24.

FIG. 26 shows a rear and left side perspective view of another alternative example of a tube holder for the device shown in FIG. 1.

FIG. 27 shows a rear view of the tube holder of FIG. 24.

FIG. 28 shows a rear and left side perspective view of another alternative example of a tube holder for the device shown in FIG. 1.

FIG. 29 shows a rear view of the tube holder of FIG. 28.

FIG. 30 shows a front and left side perspective view of the tube holder of FIG. 28 in use on a device like that in FIG. 1.

FIG. 31 shows a rear and left side perspective view of another alternative example of a tube holder for the device shown in FIG. 1.

FIG. 32 shows a rear view of the tube holder of FIG. 31.

FIG. 33 shows a front and left side perspective view of the tube holder of FIG. 31 in use on a device like that in FIG. 1.

FIG. 34 shows a rear and left side perspective view of another alternative example of a tube holder for the device shown in FIG. 1.

FIG. 35 shows a rear view of the tube holder of FIG. 34.

FIG. 36 shows a front and left side perspective view of the tube holder of FIG. 34 in use on a device like that in FIG. 1.

FIG. 37 shows a rear and left side perspective view of another alternative example of a tube holder for the device shown in FIG. 1.

FIG. 38 shows a rear view of the tube holder of FIG. 37.

FIG. 39 shows a front and left side perspective view of the tube holder of FIG. 37 in use on a device like that in FIG. 1.

### DETAILED DESCRIPTION OF THE DISCLOSURE

The disclosed ET tube holding devices solve or improve upon one or more of the above-noted and/or other problems and disadvantages with prior know holding devices. In one example, the disclosed devices have a bite block integrated into a tube holder portion of the device. In one example, the disclosed devices have a tube holder that is side-to-side adjustable along with an ET tube secured by the device. In one example, the disclosed devices have a bite block that is integrally molded as a part of the tube holder. In one example, the disclosed devices have a bite block with one or more features to assist in inserting an ET tube, retaining the ET tube in position once inserted, and accommodating an accessory line that passes within the bite block but outside of the ET tube. These and other objects, features, and advantages of the present invention will become apparent to those having ordinary skill in the art upon reading this disclosure.

Turning now to the drawings, FIGS. 1-4 show one example of a device 30 for holding and securing a tube, such as an ET tube, on a patient. Many aspects of the device are disclosed in detail in the aforementioned U.S. Pat. No. 5,490,504. The entire content of the '504 patent are hereby incorporated by reference herein. As shown in FIG. 1, the device 30 is an ET tube attachment device for securing and ET tube to a patient requiring critical medical care. The disclosed device 30 generally has a track 32 that is configured to fit adjacent a lip on a patient's face. In this particular example, the track 32 is configured to rest above the patient's upper lip. However, the track 32 can also be configured to rest below a patient's lower lip in another example. In each example, however, the track 32 is intended to extend laterally or lie horizontally across a portion of the user's face. The track 32 has a face contacting side 34 and an exposed side 36 opposite the face contacting side. The track 32 also has a pair of opposite ends 38.

The device 30 also generally has a tube holder 40 that is coupled to and slidable along the track 32 between the opposite ends 38 on the track. The device 30 also has a positioning mechanism 42 that is releasably lockable to allow selective lateral repositioning of the tube holder 40, as well as an endotracheal tube 44 that is held or secured thereby, along the track 32. The positioning mechanism 42 is also configured to retain the tube holder 40 at a selected position along the track 32. The device 30 also incorporates a bite block 46 that is carried by the tube holder 40 and slidable therewith along the track 32. Details of the bite block 46 are described in greater detail below. The bite block 46 in this example is positioned and spaced vertically below the track 32 so that the bite block extends between the teeth within a patient's mouth during use. If the track 32 were below a patient's lower lip, the bite block 46 would be spaced vertically above the track.

The track 32 is preferably made of a tough, durable, semi-rigid but somewhat flexible plastic material, such as polyethylene, and is preformed or molded with a curved or arcuate shape to fit on a region adjacent to and along one lip of the patient. Although track 32 can be positioned along either of the patient's lips, it may be preferable to position the track along the patient's upper lip to avoid having movement of the patient's jaw affect positioning and performance of the device 30, and particularly the ET tube 44.

The device 30 in this example also has a cheek plate 50 is connected to each of the opposite ends 38 of the track 32. A skin friendly cheek pad 52 is also coupled to each of the cheek plates 50 on the inside face of each plate. An adhesive layer (not show) can be provided on the face contacting side 54 of each of the cheek pads 52. The adhesive layer can also be skin friendly and can help adhere the cheek plates 50 and track 32 to the patient's face during use. The cheek plates 50 and pads 52 can be curved or contoured to closely follow the curved contour of a patient's face. Each cheek plate 50 in this example can have one or more strap loops 56 at or near their free ends. An adjustable head strap 58 can be coupled to the device 30 via the strap loops 56 for securing the device to a patient's head and retaining the track 32, cheek plates 50, and cheek pads 56 on the patient's face. A separate lip pad 58 can be provided on the face contacting side 34 of the track 32 as well. The lip pad can be adhered to the track and can also have a skin friendly adhesive on the rear exposed side to help retain the track in position against the patient's face during use.

As will be evident to those having ordinary skill in the art, the track, cheek plates, cheek pads, and head strap can vary in configuration and construction and yet fall within the scope of the invention and claims. The track and cheek plates can be molded as one integrated plastic structure, if desired. The head strap can be formed having any suitable adjustable fastening mechanism, such as a hook and loop structure on a fabric strap. The cheek plates can be formed having any number of configurations and constructions and can utilize a minimum amount of base material (i.e., plastic) and yet function as intended.

With reference to FIGS. 5-11, the tube holder 40 generally has a shuttle 60 that is slidably mounted on the track 32. The tube holder 42 also has an arm 62 that extends in a direction perpendicular to the track 32 and in a direction away from the exposed or outer surface 36 of the track. Securement means are provided on the arm 62 for securing an ET tube 44 thereto in a direction parallel to the arm. In one example, the securement means can employ a soft, flexible, elongate tube strap 64 of an elastomeric material. One end of the tube strap 64 has an enlarged retaining portion 66. As shown in FIGS. 5, 8, and 10, a slot 68 for receiving the tube strap 64 is formed across the arm 62. The slot 68 has chamfered or recessed entries 70 on either side so that the strap 64 can be threaded through the slot in either direction and the retaining portion 66 can seat in one of the chamfered entries 70, fixing that end of the strap to the arm 62. A free length of the tube strap 64 extends in a direction transverse to arm 62 and can be wrapped around the ET tube 44 as shown in FIG. 1. An adhesive pad 32(not shown) or layer, such as a suitable pressure-sensitive adhesive, can be provided on an inner surface of the tube strap 62 to further restrain the ET tube 44 from rotational or longitudinal movement when secured against the bottom of the arm 62. A plurality of sharp, nub-like tangs or spikes 72 can also be provided on the bottom surface 74 of the arm 62. The spikes 72 can impinge on the exterior surface of the ET tube 44 to frictionally and/or physically engaging the ET tube and further restraining the tube from movement.

A clamping means is provided on a top side 75 of the arm 62, opposite the bottom side 74. The clamping means in the disclosed example is substantially similar to that disclosed in the aforementioned '504 patent. The clamping means is configured to securely engage and clamp a segment of the tube strap 64 along its free length, also shown in FOIG. 1. The clamping means can maintain the tube strap 64 in tension to further help restrain the ET tube 44 from unintended movement. In the disclosed example, the clamping means is an elongate, cantilevered clamping lever 76 that is attached via a living hinge 78 to the tube holder 40, as shown in FIGS. 8 and 10. The clamping lever 76 can be pivoted and raised about the living hinge 78. The free length of the tube strap 62 can be folded over the top side 75 of the arm 6. The clamping lever 76 can be lowered and locked in place against the arm 62 to pinch and maintain the tube strap 62 in tension about the ET tube 44. A lengthwise or axial rib 80 is provided in this example and protrudes down from the clamping lever 76. The top side 75 of the arm 62 has a corresponding longitudinal or lengthwise channel or recess 82 sized to receive the rib 80 when the clamping lever is closed. The rib 80 and channel 82 are provided in order to crimp the tube strap 62 and helps to ensure that the tube strap does not slip from between the arm 62 and the clamping lever 76.

In this example, a latching means is also provided to lock and hold the clamping lever 76 in the locked or clamped condition of FIG. 1. The latching means in one example can have a flexible, resilient catch 84 provided on the top side 75 and near the front end of the arm 62. When the clamping lever 76 is lowered onto the top side 75 of the arm 62, the catch 84 will flex forward to permit the front edge 86 of the clamping lever 76 to snap past the catch 84. The catch 84 will then hold and retain the clamping lever 76 in the closed or clamped position of FIG. 1 to tightly engage the tube strap 62. To release the ET tube 44, a person can pull the catch forward away from the clamping lever 76 until the front edge 86 can clear the catch 84. The clamping lever 76 can then be raised to release the tube strap 62. This type of latching means allows one to release the ET tube 44, readjust its position, and then re-secure the tube again without having to replace any components, tape, and the like.

The arm 62 is connected to the shuttle 60 by a flexible leg 90. The flexible leg 90 in this example has one or more relieved sections 92 that allow the leg to bend and flex so that the arm 62 can move slightly relative to the shuttle. Such flexibility can impart a degree of give or yield between the shuttle and the arm so that the track can stay in position on the patient's face even while the patient's involuntary movements may cause movement of the ET tube 44 and the arm 62 during use.

As will be evident to those having ordinary skill in the art upon reading this disclosure, the disclosed device 30 is not to be limited to the particular tube holder construction disclosed herein. The arm, latching means, clamping means, flexible leg, and shuttle can vary in configuration and construction and yet function as intended.

The positioning mechanism 42 in the disclosed example is substantially similar to that disclosed in the aforementioned '504 patent. In general, the positioning mechanism has two components with one being carried on the track 32 and the other being carried on the shuttle 60 of the tube holder 40. The positioning mechanism 42 in this example allows selective lateral positioning of the tube holder 40 and the ET tube 44 along the track 32 without having to remove the device 30 from the patient or the ET tube from the device. In this example, one component of the positioning mechanism is a rail 94 disposed on the exposed side 36 of the track 32. The rail 94 is generally T-shaped in cross-section (i.e., when viewed from the side of the device 30) as shown in FIG. 2. The rail 94 is complementary shaped relative to a back side of the shuttle 60. In this example, the shuttle has a C-shaped sliding retainer 96 that slides along, captures, and engages the rail. The T-shaped rail 94 and C-shaped retainer 96 can fit snuggly to one another but without impeding lateral sliding movement of the shuttle 60 along the track 32. One or more stops (not shown) may be provided at each of the opposite ends 38 of the track 32 to keep the shuttle 60 from sliding off either end of the rail 94 during use. These stops can allow the shuttle to be snapped onto one end of the track 32 during assembly of the device 30.

The positioning mechanism also includes a locking means, in this example also partly on the shuttle 60 and partly on the track 32, to positively lock and hold the tube holder 40 and ET tube 44 in the selected position of adjustment on the track. In one example, as shown in FIGS. 1-4, the locking means has a pair of dogs or flex fingers 100 oriented generally perpendicular to the track 32. The flex fingers 100 are resiliently connected to the shuttle 60 and have prongs 102 that project rearward toward the track 32. The prongs 102 are positioned to engage any one of a plurality of ratchet teeth 104 provided on a front face of the rail 94. When the flex fingers 100 are in an unflexed condition, the prongs 102 seat in recesses 106 between the teeth 104. Grip ends 108 on the flex fingers 100 can be squeezed together, which spreads the flex fingers outward away from one another and disengages the prongs 102 from the ratchet teeth 104. When disengaged, the tube holder 40 and shuttle 60 can slide along the rail 94 to a desired position along the track 32. This can be done to allow medical personnel to access the patient's mouth without having to remove the device 30 or the ET tube 44. When pressure on the grip ends 108 the flex fingers 100 is released, the prongs 102 will return and engage the ratchet teeth 104 to retain the tube holder 40 in the selected position along the track 32.

As will be evident to those having ordinary skill in the art, the positioning mechanism components, including the locking means, the rail, and the shuttle retainer, can vary from the example shown and described herein. The details of the structure and function of these aspects of the device 30 can be varied and yet function as intended. The device 30 has a tube holder 40 that is side-to-side adjustable along the track, even when the device is attached to a patient. Thus, the ET tube 44 can be move from one side to another within the patient's mouth to allow medical personnel to clean the patient's teeth or attend to other medical care without having to completely remove the device 30 from the patient and without having to remove the ET tube 44 from the device. This makes caring for the patient simpler and easier.

According to the teachings of the present invention, the bite block 46 is integrated into the tube holder, as best illustrated in FIGS. 5-11. In one example, the bite block can be integrally molded from a suitable plastic or similar material as a part of the tube holder 40. This can render the entire tube holder 40 a one-piece unit inclusive of the bite block 46, the arm 62, the shuttle 60, the flex fingers 100, and the clamping mechanism for the tube strap. In one alternate example, the bite block 46 could be a separate element that is configured to attach to the tube holder. This could be done utilizing a complementary snap connection between bite block and holder or utilizing an adhesive, fasteners, or the like. In such an example, the bite block could be utilized on a patient where the medical personnel determine such usage beneficial. The bite block could also be removed in such an example if the medical personnel determined that the bite block should not be used for some reason.

In the disclosed example, the bite block 46 is molded to a rear end 110 of the arm 62 opposite the catch 84. A molded joint 112 connects the bite block 46 to the arm 62 in this example. The molded joint 112 can vary in configuration and construction. The intent is for the connection to be robust and durable so that the bite block 46 remains attached to the tube holder 40, as long as intended. The joint 112 can also be positioned and configured to connect the bite block 46 to the tube holder 40 at a number of different locations on both the bite block and holder. In this example, the molded joint 112 is on the rear end 110 of the arm 62 and connects to a forward end 114 of the bite block 46. Also in this example, the bite block 46 is positioned spaced vertically below the track 32. In other configurations, as noted above, it is possible that the bite block be positioned and spaced above the track. The positioning of the bite block 46 in this example is such that the ET tube 44 can still be retained in place by the tube strap 64 and by the spikes 72, which are still exposed forward of the bite block on the bottom side 74 of the arm 62.

Features of the bite block 46 are depicted in FIGS. 5, 6, and 8. The bite block 46 has a generally tubular wall 120 having a substantially cylindrical shape with a length and a central axis. A central opening 122 extends along the length of the bite block 46 and the wall 120 has a pair of opposite open ends including the aforementioned forward end 114 and a rear or working end 126. The working end portion of the wall 120 of the bite block extends in the direction of the face contacting side 34 of the track 32, i.e., in a rearward direction and extends further rearward beyond the surface or face of the face contacting side.

In the disclosed example, the bite block 46 has an insertion slot 128 through the wall 120. The insertion slot 128 extends along the entire length of the bite block 46. The wall 120 can be formed of a suitable plastic material so that the bite block 46 is sufficiently flexible to allow an ET tube 44 to be inserted laterally into the central opening 122 through the insertion slot 128. The flexibility of the wall 120 can allow the width of the slot 128, and thus the diameter of the wall, to expand when inserting the tube and then to spring back to the normal at rest slot width and wall diameter once the tube is fully inserted. The insertion slot 128 is defined between spaced apart free edges 130 (upper) and 132 (lower) of the wall 120. The free edges 130, 132 confront one another along the length of the bite block 46 and define the width of the slot 128. The wall 120 also has a blind slot 134 that is formed in the working end portion of the bite block 46. The blind slot 134 is open at the working end 124 of the wall 120 and terminates only part way along the wall at a closed or blind end 136. Thus, unlike the insertion slot 128, the blind slot 134 extends only part way along the length of the wall 120. The blind slot is also positioned generally opposite the insertion slot 128 in the wall. The blind slot 134 can add some resilience and flexibility to the wall 128. Thus, during use, the wall 120 can give slightly if a patient were to occasionally exert a great amount of force upon the bite block 46 during use. This would divert some of the load or absorb some of the energy from such a force through the wall 120 instead of directly to the patient's jaw and/or teeth.

The bite block 46 can also include one or more features to accommodate accessory lines passing through the bite block adjacent the ET tube 44. Such an accessory line channel can extend the length of the bite block and be formed between the wall and the ET tube within the central opening of the bite block. In this example, the bite block 46 has an accessory line channel 138 that extends the length of the bite block 46 within the central opening 122. The accessory line channel 138 can be formed, at least in part on an inner surface 140 of the wall 120. The accessory line channel 138 can also be formed in part by the outer surface of the ET tube 44, depending on the configuration and construction of the channel. In this example, the accessory line channel 138 is formed between the inner surface 140 of the wall 120 and the outer surface of the ET tube 44. Two spaced apart ribs 142 extend along the length of the bite block 46 on the inner surface 140 of the wall 120. The channel would be defined between the ribs and between the outer ET tube surface and the inner surface 140 of the wall 120. The ribs 142 create a space between the inner surface 140 and the ET tube 44. An accessory line, such as a pilot tube 144 (see FIG. 1) for inflating a retention cuff or balloon (not shown) on the end of the ET tube, can be run along the outside of the ET tube 44 and through the bite block 46 along the channel 138 without being pinched off, closed, or crushed.

More than one accessory line channel can be formed along the inside of the bite block 46. Three or more spaced apart ribs can be provided to define two or more separate channels, if desired. Also, two or more accessory lines can be run along the same channel, if desired. The accessory lines can provide any type of additional line function needed to treat a patient, including but certainly not limited to the pilot line 144. Also, the configuration, construction, and performance features of the bite block can vary from the bite block 46 described above. For example, the free edges 130, 132 of the insertion slot 128 on the bite block 46 can be varied in shape and contour so as to help the wall 120 retain a generally cylindrical shape, even when a patient's teeth exert a substantial crushing force on the bite block during use. The upper free edge 130 could have a first contoured shape and the lower free edge 132 could have a corresponding second contoured shape configured to close against and engage the first contoured shape if a sufficient clamping force is exerted on an outer surface of the bite block.

For example, FIGS. 12 and 13 show a tube holder 150 that is substantially identical to the earlier described tube holder 40. In this example, the tube holder 150 has a bite block 152 that also has a tubular shape with a substantially cylindrical wall 154. However, the bite block 152 has a simpler configuration with no accessory line channel and no blind slot. The bite block 152 does have an insertion slot 156 along a length of the slot and through the wall 154. Free edges 158, 160 of the insertion slot 156 are contoured in this example and are closer together resulting in a narrower insertion slot 156 than the slot 128 of the bite block 46. The upper free edge 158 has an angled or beveled edge forming a flat face 162 and the lower free edge 160 has a corresponding angled or beveled edge defining a complementary flat face 164. If a patient were to apply sufficient bite force on the bite block, the flat faces 162, 164 would engage and mate with one another under less of a load than the bite block 46 because the free edges 158, 160 are closer together. Also, the flat faces 162, 164 being in contact will help prevent the free edges 158, 160 from bypassing one another and thus will help prevent the wall 154 from collapsing beyond its cylinder shape.

FIGS. 14 and 15 show another example of a tube holder 170 that is also substantially identical to the earlier described tube holder 40. In this example, the tube holder 170 has a bite block 172 that also has a tubular shape with a substantially cylindrical wall 174. The bite block 172 also has a simpler configuration with no accessory line channel and no blind slot in comparison to the bite block 46. The bite block 172 does have an insertion slot 176 along a length of the slot and through the wall 174. Free edges of the insertion slot 176 are also contoured in this example. Forward edge portions 178a, 180a are not contoured and are further apart from one another leaving a greater slot width at a forward end of the insertion slot 176. Rearward edge portions 178b, 180b of the free edges are closer together resulting in a narrower width slot over part of the insertion slot 176 than the slot 128 of the bite block 46.

The upper free edge portion 178b has an angled or beveled edge forming a flat face 182 and a convex V-shaped terminus 184 along the free edge portion. The lower free edge portion 180b has a corresponding angled or beveled edge defining a complementary flat face 186 and also has a stepped ridge 188 protruding into the central opening 122. The stepped ridge cooperates with the complementary flat face 186 to create a concave V-shaped trough or groove 190 along the free edge portion 180b. If a patient were to apply sufficient bite force on the bite block 170, the bottom terminus 184 would engage and seat in the groove 190 to help prevent the two free edges 178b, 180b from bypassing one another and thus to help prevent the wall 172 from collapsing beyond its cylinder shape. The contoured free edge portions in this example may be more robust in this regard that those of the bite block 150.

FIGS. 16 and 17 show another example of a tube holder 200 that is also substantially identical to the earlier described tube holder 40. In this example, the tube holder 200 has a bite block 202 that is substantially the same as the bite block 170, other than the contoured edge portions. In this example, the bite block 202 also has a tubular shape with a substantially cylindrical wall 204. The bite block 202 also has no accessory line channel and no blind slot in comparison to the bite block 46. The bite block 202 does have an insertion slot 206 along a length of the slot and through the wall 204. Free edges of the insertion slot 206 are also contoured in this example. Forward edge portions 208a, 210a are not contoured and are further apart from one another leaving a greater slot width at a forward end of the insertion slot 206. Rearward edge portions 208b, 210b of the free edges are closer together resulting in a narrower width slot over part of the insertion slot 206 than the slot 128 of the bite block 46.

The upper free edge portion 208b has a rounded convex contour forming a rounded face 212 along the free edge portion. The lower free edge portion 210b has a flange 214 that is wider than the wall 204 thickness. The upward facing side of the flange 214 has a concave rounded groove sized to complement the rounded face 212 of the upper edge portion 208b. If a patient were to apply sufficient bite force on the bite block 200, the rounded face 212 would engage and seat in the rounded groove 216 to help prevent the two free edges 208b, 210b from bypassing one another and thus to help prevent the wall 202 from collapsing beyond its cylinder shape. The contoured free edge portions in this example also may be more robust in this regard that those of the bite block 150.

The bite block in other examples can have alternative concave and convex complementary shape, or other complementary contoured shapes that can engage and mate with one another. The invention is not limited only to these examples described herein.

For example, FIGS. 18 and 19 show a tube holder 220 that is also substantially identical to the earlier described tube holder 40. In this example, the tube holder 220 has a bite block 222 that also has a tubular shape with a substantially cylindrical wall 224. The bite block 222 has a simpler configuration with no blind slot in comparison to the tube holder 46. The bite block 222 does have an insertion slot 226 along a length of the slot and through the wall 224. Free edges 228, 230 of the insertion slot 226 are contoured in this example and are again closer together resulting in a narrower insertion slot 226 than the slot 128 of the bite block 46. Both the upper free edge 228 and lower free edge 230 have mirror image flanges 232, 234 that extend radially inward into the central opening of the bite block 220.

The flanges 232, 234 make the free edges 228, 230 wider than the wall 224 thickness. The flanges 232, 234 are configured to close against and engage one another when a sufficient clamping force is exerted on the bite block, which is less than that of the bite block 46, again because the free edges 228, 230 are closer together. Also, the flanges 232, 234 being wider than the wall 224 thickness will help prevent the free edges 228, 230 from bypassing one another and thus will help prevent the wall 224 from collapsing beyond its cylinder shape. Each of the flanges 232, 234 in this example also creates an accessory line channel 236, 238 along an inner surface 240 of the wall 224 adjacent the flanges within the central opening of the bite block 220. It is possible that only one of the flanges is configured to form or define such an accessory line channel.

FIGS. 20 and 21 show another alternate example of a tube holder 250 that is substantially identical to the earlier described tube holder 40. In this example, the tube holder 250 has a bite block 252 that also has a tubular shape with a substantially cylindrical wall 154. The bite block 252 also has no accessory line channel and no blind slot. The bite block 252 does have an insertion slot 256 along a length of the slot and through the wall 254. Free edges 258, 260 of the insertion slot 256 are spaced apart and contoured in a manner that is essentially identical to the free edges 158, 160 on the bite block 152 of the tube holder 150 described previously. The bite block 250 also has a living hinge 266 or relief in the wall 254 generally opposite the insertion slot 256 and extending a length of the bite block. The living hinge can be formed in a thickened portion 268 of the wall as shown. Alternatively, the entire wall can be thicker, other than the living hinge 266. The living hinge can be provided to allow for easier flexing of the wall to insert the ET tube. The thicker wall material can provide a more robust bite block while the living hinge still permits easy ET tube insertion via the insertion slot.

FIGS. 22 and 23 show yet another example of a tube holder 270 with features similar to earlier described bite block features. In this example, the tube holder 270 has a bite block 272 with two sets of ribs 274 forming two accessory line channels, one above the ET tube and one below the ET tube. The ribs 274 are otherwise similar to the ribs 142 described previously. The bite block 272 also has a living hinge 276 similar to the living hinge 266 in the previous example. Also in this example, free edges 278 of the bite block 272 along a insertion slot 279 can be configured to contact one another upon compression of the bite block. In this example, the free edges 278 can also be configured so that one of the free edges slips past the other until it contacts one of the ribs 274, which can act as a stop to prevent further compression.

FIGS. 24 and 25 show still another alternate example of a tube holder 280 with features similar to earlier described bite block features. In this example, the tube holder 280 has a bite block 282 with an insertion slot 284 similar to the slot 128 of the bite block 46 with a wide slot width. This bite block 282 has no blind slot opposite the insertion slot 284 but does have two spaced apart ribs 286 forming a single accessory line channel within the bite block.

FIGS. 26 and 27 show still another alternate example of a tube holder 290 with most features similar to earlier described bite block features. In this example, the tube holder 290 has a bite block 292 with an insertion slot 294 similar to the slot 128 of the bite block 46 with a wide slot width. This bite block 282 also has a blind slot 296 similar to the blind slot 134 on the bite block 46 and positioned opposite the insertion slot 294. The bite block 292 has a groove 298 formed along a length of the bite block and recessed into an inner surface 300 of the bite block wall 302. The groove 298 creates an accessory line channel that recessed into the wall 302, instead of using protrusions to create space between the inner wall and an ET tube in the bite block, as in prior examples. The groove can be V-shaped in cross-section or can have a rounded, semispherical, or other shape as well.

FIGS. 28-30 show yet another example of a tube holder 320 that is nearly identical to the tube holder 270 described above. In this example, the tube holder 310 has a bite block 322 with two sets of ribs 324 and 326 forming two accessory line channels, one above the ET tube and one below the ET tube. In this example, the upper ribs 326 are longer than the lower ribs 324. The longer upper ribs provide a deeper accessory line channel between them. In one example, as shown in FIG. 30, the accessory line can be a subglottic suction line 328 that communicates with the ET tube. Such a tube may have a larger diameter than a conventional pilot line and require a deeper channel. When the bite block 322 is compressed during use, the rib length can determine how much crush is imparted on the accessory lien, such as the subglottic suction line.

FIGS. 31-33 show still another example of a tube holder 330 that is substantially the same as many of the prior described example. In this example, the tube holder 330 has a bite block 332 that is substantially the same as the bite block 46. However, the bite block has a second scallop or blind slot 334 on the front end of the bite block and opposite the insertion slot 336. The tube holder 330 also has a revised joint 336 that extends the bite block 332 a bit more rearward from the arm 340 than prior described tube holders. This extended joint 336 in combination with the blind slot 338 creates a gap or clearance 342 under the arm and forward of the bite block 332. As shown in FIG. 33, a second accessory line 344, such as a subglottic suction line, can exit the ET tube and pass under the arm 340 and joint 336.

FIGS. 34-36 show still another example of a tube holder 350 that is substantially the same as many of the prior described example. In this example, the tube holder 350 has a bite block 352 that has a raised recess or channel 354 formed in the upper surface of and extending the length of the bite block 352. The channel 354 create a larger sized passage within the bite block 352 for an accessory line without significantly affecting the size of ET tube that the bite block 352 can handle. As shown in FIG. 36, a second accessory line 356, such as a subglottic suction line, can exit the ET tube and pass within the channel 354 along with the ET tube.

FIG. 37 shows another example of a tube holder 360 that is essentially the same as the tube holder 40 described earlier and has a substantially similar bite block 362. However, in this example, the bite block 362 has a softer pad 364 provided on the top and bottom of the bite block 362. The pads can be adhered to the exterior of the bite block or can be in-molded or dual molded with the bite block but from a softer material. The soft pads 364 can be added for patient comfort and perhaps safety. The forces applied to the bite block 362 during use would be at least partly dispersed across the surface of the block via the softer pads 354. This can minimize stress concentration on the barrel of the bite block and help reduce the likelihood of damage to the patients dentin while also increasing comfort for those that are awake and actively biting. In another example, the pads 364 could be combined with the taller bite block 352 on top of the channel 354 from the immediately prior example to increase comfort for the patient. The soft material pads 364 in such an example could be over-molded to cover the raised area of the channel 354 for the subglottic suction line 356 or other accessory line to create a soft landing for the patient's upper incisors.

In still another example as shown in FIGS. 38 and 39, a tube holder 366 that is substantially the same as the earlier described bite block 270 of FIGS. 23 and 24, with shorter upper ribs 367 can be used, but have an increased diameter bite block 368. The tube holder 366 can be used in conjunction with a larger sized accessory line 370, such as a subglottic suction tube. The ribs 367 and line 370 can be configured to allow for a predetermined amount of compression or crush on the line. The bite block 368 can also be configured to allow for a predetermined amount of compression or crush on the ET tube 372. A shown in FIG. 39, and as discussed above, the bite block 368 can be configured to allow for some compression before the free ends 374 along the insertion slot 376 come in contact with one another to resist further compression. The live hinge 378, similar to the earlier described hinges can help with flexibility in the bite block 368 to insert the ET tube 372 and can also help with allowing some limited compression without much resistance.

Any number of the bite block and tube holder features described above can be used in combination, even though such combination is not specifically mentioned herein. Also, one or more features can also be added to any of the bite block examples disclosed herein to aid in inserting an ET tube into the insertion slot and to help prevent discomfort and irritation to the patient during use. As shown in FIG. 10 for example, the forward ends (or the rear ends, if desired) of the free edges 130, 132 on the bite block 46 can include angled or tapered entry segments 310, 312 at the leading edge of the insertion slot 128. 2 The tapered entry segments 310, 312 can make the insertion slot 128 wider, but only at the forward end of the bite block 46. This can make it easier for medical personnel to spread apart the free edges 130, 132 in order to aid in starting to insert an ET tube 44 into the bite block 46. Such a feature can be included on any of the disclosed bite blocks and is shown on many of them herein.

In another example, the end face of the bite block need not lie in a plane. Instead, the end face can be curved to form rounded curved contours, especially on the working end of the bite block that will lie within a patient's mouth. For example, FIGS. 5 and 10 show that the bite block 46 has curved contoured regions 314 in the axial direction at the working end 124 of the bite block 46. Likewise, the exposed end edges of the bite block can also be smooth and curved or rounded. For example, an end edge 316 of the bite block 200 shown in FIGS. 16 and 17 are smooth and rounded instead of having sharp corner, particularly at the working end of the bite block. Such features can make the device 30 more comfortable for a patient during use.

The disclosed ET tube holding device 30 can be applied or installed on the patient with the ET tube 44 already positioned in the patient's mouth and trachea. If a temporary bite block device is already prepositioned about the tube, that bite block can be removed and the bite block as described herein can be attached to the ET tube at the same time that the device 30 is installed. The disclosed bite blocks can be constructed from materials and material thicknesses and with features that render the bite block sufficiently rigid to inhibit the inserted ET tube from being crushed or closed off by a patient's bite during use and yet sufficiently flexible to allow relatively easy insertion and removal of the ET tube as needed.

Although certain ET tube holding devices, features, components, and methods have been described herein in accordance with the teachings of the present disclosure, the scope of coverage of this patent is not limited thereto. On the contrary, this patent covers all embodiments of the teachings of the disclosure that fairly fall within the scope of permissible equivalents.

What is claimed is:

1. A device for holding an endotracheal tube to a patient, the device comprising:
a track configured to fit adjacent a lip on a patient's face, the track having a face contacting side, an exposed side opposite the face contacting side, and a pair of opposite ends;
a tube holder coupled to and slidable along the track between the opposite ends;
a positioning mechanism that is releasably lockable to allow selective lateral repositioning of the tube holder and an endotracheal tube held thereby along the track and to retain the tube holder at a selected position along the track; and
a bite block carried by the tube holder and slidable therewith along the track, the bite block having a tubular wall with a generally cylindrical shape, a central opening along a length of the bite block, and a pair of opposite open ends,
wherein the bite block is positioned spaced vertically from the track and has a working end portion that extends in the direction of and further beyond the face contacting side of the track.

2. A device according to claim 1, wherein the bite block has an insertion slot through the wall and along the entire length of the bite block, the wall being sufficiently flexible to allow an endotracheal tube to be inserted laterally into the central opening through the insertion slot.

3. A device according to claim 2, wherein the insertion slot is defined between spaced apart free edges of the wall that confront one another along the length of the bite block.

4. A device according to claim 3, wherein one of the free edges has a first contoured shape and the other of the free edges has a corresponding second contoured shape configured to close against and engage the first contoured shape if a sufficient clamping force is exerted on an outer surface of the bite block.

5. A device according to claim 4, wherein the first contoured shape is a convex rounded edge and the second contoured shaped is a concave rounded edge sized to receive the convex rounded edge.

6. A device according to claim 4, wherein the first contoured shape is an angled or beveled edge and the second contoured shape is a corresponding angled or beveled edge.

7. A device according to claim 4, wherein the first contoured shaped is a convex V-shaped protrusion and the second contoured shape is a corresponding concave V-shaped groove.

8. A device according to claim 3, wherein each of the free edges has a flange extending inward into the central opening of the bite block and configured to close against and engage one another if a sufficient clamping force is exerted on an outer surface of the bite block.

9. A device according to claim 8, further comprising an accessory line channel formed along an inner surface of the wall adjacent one or both of the flanges within the central opening of the bite block.

10. A device according to claim 2, further comprising a living hinge or relief in the wall generally opposite the insertion slot and extending a length of the bite block.

11. A device according to claim 2, further comprising a blind slot formed in the working end portion of the bite block and extending only part way along the length of the wall and positioned generally opposite the insertion slot in the wall.

12. A device according to claim 1, further comprising an accessory line channel extending the length of the bite block and formed between an inner surface of the wall and an outer surface of an endotracheal tube positioned within the central opening of the bite block.

13. A device according to claim 12, wherein the accessory line channel is formed between two spaced apart ribs extending the length of the bite block on the inner surface of the wall.

14. A device according to claim 12, wherein the accessory line channel is formed as a V-shaped groove extending along and recessed into the inner surface of the wall.

15. A device according to claim 1, wherein the track has a lip pad on the face contacting side, the track is configured to rest above an upper lip of a patient, and the bite block is spaced below the track.

16. A device according to claim 1, further comprising:
a cheek plate connected to each of the opposite ends of the track;
a cheek pad coupled to each of the cheek plates;
an adhesive layer on a face contacting side of each pad; and
an adjustable head strap coupled to the device for securing the device to a patient's head and retaining the track, cheek plates, and cheek pads on the patient's face.

17. A device according to claim 1, the tube holder further comprising a tube strap configured to wrap around and secure an endotracheal tube to the tube holder.

18. A device according to claim 17, wherein a surface of the tube strap includes an adhesive that contacts an outer surface of an endotracheal tube when the tube strap is wrapped around the endotracheal tube.

19. A device according to claim 1, further comprising retention spikes protruding from a surface of the tube holder and positioned to contact and impinge on an outer surface of an endotracheal tube when attached to the tube holder.

20. An endotracheal tube securing device comprising:
a track configured to fit above an upper lip on a patient's face, the track having a face contacting side, an exposed side opposite the face contacting side, and a pair of opposite ends;
a tube holder coupled to and slidable along the track between the opposite ends;
a positioning mechanism that is releasably lockable to allow selective lateral repositioning of the tube holder and an endotracheal tube held thereby along the track and to retain the tube holder at a selected position along the track;
an adjustable head strap coupled to the device for securing the device to a patient's head and retaining the track on the patient's face;
a cheek plate connected to each of the opposite ends of the track, each cheek plate carrying a cheek pad with an adhesive layer on a face contacting side of the cheek pad; and
a bite block integrally molded as part of the tube holder and slidable therewith along the track, the bite block having
a tubular wall with a generally cylindrical shape,
a central opening along a length of the bite block, and a pair of opposite open ends, and
an insertion slot through the wall and along the entire length of the bite block,
wherein the bite block is positioned spaced vertically from the track and has a portion extending further rearward than a plane of the face contacting side of the track.
